(19) United States
(12) Patent Application Publication
(10) Pub. No.: US 2023/0132333 A1
(43) Pub. Date: Apr. 27, 2023

(54) Methods of Calibrating a Blood Flow Monitoring Device, Methods of Monitoring Blood Flow, and Blood Flow Monitoring Systems

(71) Applicant: Cook Medical Technologies LLC, Bloomington, IN (US)

(72) Inventors: Suresh Dwarakaprasad, Medford, NJ (US); Travis Dillon, Winston-Salem, NC (US); Fionan Keady, Glenamaddy, Co. Galway (IE); David Halleck, Frederick, CO (US)

(73) Assignee: Cook Medical Technologies LLC, Bloomington, IN (US)

(21) Appl. No.: 17/967,930

(22) Filed: Oct. 18, 2022

Related U.S. Application Data
(60) Provisional application No. 63/271,470, filed on Oct. 25, 2021.

(51) Int. Cl. A61B 5/021; A61B 5/00

(52) U.S. Cl. CPC A61B 5/021; A61B 5/743; A61B 2562/0204; A61B 2560/0238

(57) ABSTRACT

An example method of calibrating a blood flow monitoring device includes positioning a remote device and a blood flow monitor within physical proximity of one another, placing the remote device in a calibration mode such that a microphone of the remote device is active and can receive Doppler sounds emitted by a speaker of the blood flow monitor; emitting Doppler sounds through the speaker of the blood flow monitor; receiving the Doppler sounds as received Doppler sounds using the microphone of the remote device; emitting the received Doppler sounds through a speaker of the remote device as remote device sounds; receiving the remote device sounds using the microphone of the remote device; identifying any differences between the received Doppler sounds and the received remote device sounds; and calibrating the remote device based on the identified differences between the received Doppler sounds and the received remote device sounds.

### RELATED APPLICATIONS

This application claims the benefit of U.S. Provisional Application No. 63/271,470, which was filed on Oct. 25, 2021. The entire disclosure of this related application is hereby incorporated by reference into this disclosure.

### FIELD

The disclosure relates generally to blood flow monitoring. More particularly, the disclosure relates to methods of calibrating a blood flow monitoring device, methods of monitoring blood flow, and blood flow monitoring systems.

### BACKGROUND

Conventional blood flow monitors include speakers and a single bar graph (e.g., light emitting diode (LED) bar graph) to provide feedback to a clinician relating to the real-time velocity of blood flow through a blood vessel. The bar graph provides a visual indication of blood flow. The speakers provide an audible indication of blood flow through the vessel, such as by emitting sounds based on Doppler ultrasound. The quality of the sounds emitted from the speakers are device dependent and can vary depending on the type and/or model of the blood flow monitor being used. Experienced clinicians develop an ability to distinguish between adequate and inadequate blood flow through a monitored blood vessel by listening to the sounds emitted by the blood flow monitor.

Blood flow through a body vessel is monitored during a variety of procedures. For example, when a tissue flap is being implanted, blood flow is monitored to confirm blood flow through a blood vessel included in the tissue flap, which is critical to the health of the tissue flap. For these procedures, a clinician typically monitors blood flow by being physically present with the patient and the blood flow monitor, listening to the audible indication of blood flow emitted by the speaker of the monitor. Establishment of adequate blood flow can be critical to the success of many procedures, such as graft and flap placements, requiring regular and frequent monitoring of blood flow through the relevant vessels. As a result, post-operative regimens for these procedures often include blood flow monitoring every fifteen minutes for the first hour, every thirty minutes for the second hour, every hour for the next ten hours, and every four hours thereafter. Using conventional blood flow monitors requires the clinician to be physically present with the patient and the monitor at each of these monitoring intervals, which creates significant demand on their time and blocks their ability to perform other procedures that cannot be performed within the limited windows of time left available by the monitoring regimen.

A need exists, therefore, for new and useful methods of calibrating a blood flow monitoring device, methods of monitoring blood flow, and blood flow monitoring systems.

### Summary of Selected Example Embodiments

Various example methods of calibrating a blood flow monitoring device are described herein.

An example method of calibrating a blood flow monitoring device includes positioning a remote device and a blood flow monitor within physical proximity of one another, the remote device has a microphone and a speaker, the blood flow monitor has a speaker and is configured to emit Doppler sounds; placing the remote device in a calibration mode such that the microphone of the remote device is active and can receive Doppler sounds emitted by the speaker of the blood flow monitor; emitting Doppler sounds through the speaker of the blood flow monitor; receiving the Doppler sounds as received Doppler sounds using the microphone of the remote device; emitting the received Doppler sounds through the speaker of the remote device as remote device sounds; receiving the remote device sounds using the microphone of the remote device; identifying any differences between the received Doppler sounds and the received remote device sounds; and calibrating the remote device based on the identified differences between the received Doppler sounds and the received remote device sounds.

Another example method of calibrating a blood flow monitoring device includes placing a tissue flap at a point of treatment, the tissue flap includes a section of tissue that includes a blood vessel; attaching a sensor to the tissue flap to monitor blood flow through the blood vessel, the sensor is attached to the tissue flap such that it contacts the blood vessel; attaching the sensor to a blood flow monitor that has a speaker and is configured to emit Doppler sounds; activating the blood flow monitor such that the blood flow monitor is monitoring blood flow through the blood vessel; positioning a remote device and the blood flow monitor within physical proximity of one another, the remote device has a microphone and a speaker; placing the remote device in a calibration mode such that the microphone of the remote device is active and can receive Doppler sounds relating to the blood flow through the blood vessel emitted by the speaker of the blood flow monitor; emitting Doppler sounds relating to the blood flow through the blood vessel through the speaker of the blood flow monitor; receiving the Doppler sounds relating to the blood flow through the blood vessel as received Doppler sounds using the microphone of the remote device; emitting the received Doppler sounds through the speaker of the remote device as remote device sounds; receiving the remote device sounds using the microphone of the remote device; identifying any differences between the received Doppler sounds and the received remote device sounds; and calibrating the remote device based on the identified differences between the received Doppler sounds and the received remote device sounds.

Various example methods of monitoring blood flow are described herein.

An example method of monitoring blood flow includes calibrating the sound quality of a remote device that has a speaker and a microphone; placing a tissue flap at a point of treatment, the tissue flap includes a section of tissue that includes a blood vessel; attaching a sensor to the tissue flap to monitor blood flow through the blood vessel, the sensor is attached to the tissue flap such that it contacts the blood vessel; attaching the sensor to a blood flow monitor that has a speaker and is configured to emit Doppler sounds; activating the blood flow monitor such that the blood flow monitor is monitoring blood flow through the blood vessel; monitoring the blood flow through the blood vessel over a period of time; positioning the remote device at a first location that is different from a second location at which the blood flow monitor is disposed; emitting Doppler sounds relating to the blood flow through the blood vessel through the speaker of the remote device; determining if the Doppler sounds relating to the blood flow through the blood vessel indicate intervention is required, if intervention is required the method further comprises performing treatment to accomplish the intervention, if intervention is not required the method further comprises removing the sensor from the tissue flap.

Various example blood flow monitoring systems are described herein.

An example blood flow monitoring system includes a remote device and a blood flow monitor. The remote device is calibrated using a method of calibrating a blood flow monitoring device that includes positioning the remote device and a blood flow monitor within physical proximity of one another, the remote device has a microphone and a speaker, the blood flow monitor has a speaker and is configured to emit Doppler sounds; placing the remote device in a calibration mode such that the microphone of the remote device is active and can receive Doppler sounds emitted by the speaker of the blood flow monitor; emitting Doppler sounds through the speaker of the blood flow monitor; receiving the Doppler sounds as received Doppler sounds using the microphone of the remote device; emitting the received Doppler sounds through the speaker of the remote device as remote device sounds; receiving the remote device sounds using the microphone of the remote device; identifying any differences between the received Doppler sounds and the received remote device sounds; and calibrating the remote device based on the identified differences between the received Doppler sounds and the received remote device sounds.

Additional understanding of these example methods of calibrating a blood flow monitoring device, methods of monitoring blood flow, and blood flow monitoring systems can be obtained by review of the detailed description, below, and the appended drawings.

### BRIEF DESCRIPTION OF THE DRAWINGS

FIG. 1 is a schematic illustration of an example method of calibrating a blood flow monitoring device.

FIG. 2 an elevation view of a blood flow monitoring system that includes a blood flow monitor and a remote device. The remote device is positioned within physical proximity of a blood flow monitor.

FIG. 3 is a schematic illustration of another example method of calibrating a blood flow monitoring device.

FIG. 4 is a partial perspective view of a sensor attached to a tissue flap.

FIG. 5 a partial elevation view of a remote device positioned within physical proximity of a blood flow monitor. The sensor is attached to the monitor using an extension cable and the monitor is in the on state.

FIG. 6 is a schematic illustration of an example method of monitoring blood flow.

### DETAILED DESCRIPTION OF SELECTED EXAMPLES

The following detailed description and the appended drawings describe and illustrate various example methods of calibrating a blood flow monitoring device, methods of monitoring blood flow, and blood flow monitoring systems. The description and illustration of these examples are provided to enable one skilled in the art to practice a method of calibrating a blood flow monitoring device, a method of monitoring blood flow, and to make a blood flow monitoring system. They are not intended to limit the scope of the claims in any manner. The invention is capable of being practiced or carried out in various ways and the examples described and illustrated herein are merely selected examples of the various ways of practicing or carrying out the invention and are not considered exhaustive.

Various methods of calibrating a blood flow monitoring device and methods of monitoring blood flow are described herein. While the methods described herein are shown and described as a series of acts, it is to be understood and appreciated that the methods are not limited by the order of acts, as some acts may, in accordance with these methods, occur in the order shown and/or described, in different orders, concurrently with other acts described herein, or be omitted.

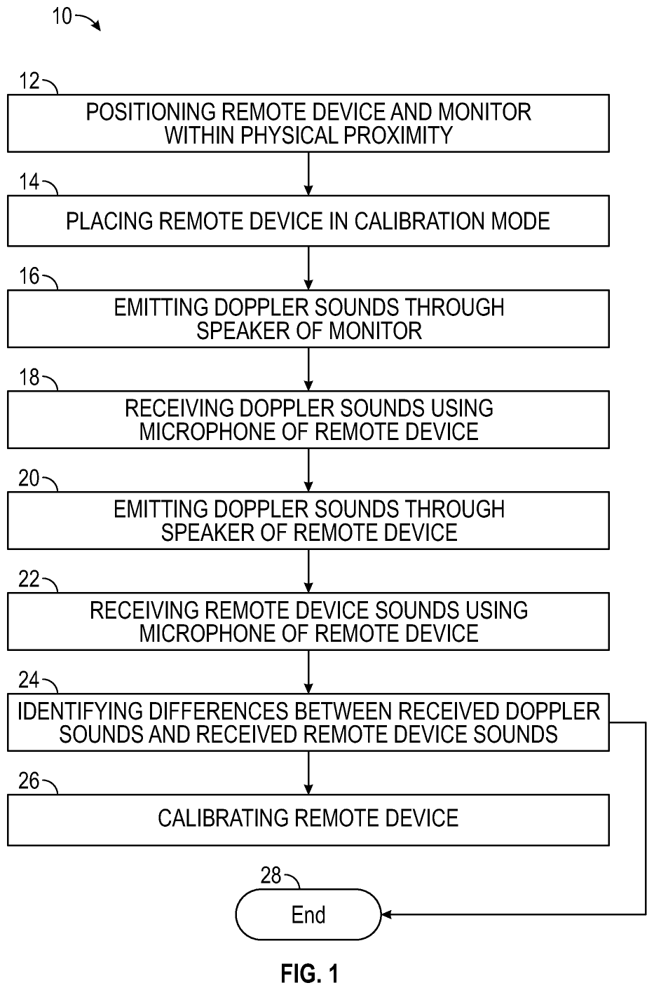

FIG. 1 is a schematic illustration of an example method 10 of calibrating a blood flow monitoring device.

A step 12 comprises positioning a remote device and a blood flow monitor within physical proximity of one another. The remote device has microphone and a speaker. The blood flow monitor has a speaker and is configured to emit Doppler sounds. Another step 14 comprises placing the remote device in a calibration mode such that the microphone of the remote device is active and can receive Doppler sounds emitted by the speaker of the blood flow monitor. Another step 16 comprises emitting Doppler sounds through the speaker of the blood flow monitor. Another step 18 comprises receiving the Doppler sounds as received Doppler sounds using the microphone of the remote device. Another step 20 comprises emitting the received Doppler sounds through the speaker of the remote device as remote device sounds. Another step 22 comprises receiving the remote device sounds using the microphone of the remote device. Another step 24 comprises identifying any differences between the received Doppler sounds and the received remote device sounds. If differences exist, another step 26 comprises calibrating the remote device based on the identified differences between the received Doppler sounds and the received remote device sounds. If no differences exist, another step 28 comprises completing the method.

Step 12 can be accomplished by positioning the remote device (e.g., a blood flow monitoring device) at any suitable distance relative to the blood flow monitor while being within physical proximity of the blood flow monitor. Examples of distances considered suitable between a remote device and a blood flow monitor include distances in which a microphone of a remote device can receive sounds emitted by a speaker of a blood flow monitor, distances equal to, greater than, less than, or about 1 foot, 2 feet, between about 0 feet and about 3 feet, and any other distance considered suitable for a particular embodiment.

A remote device can be any device that can be moved to a location that is remote relative to a blood flow monitor (e.g., a different room, a different building, a different city, state, or country), includes a microphone, a speaker, and any software and/or components capable of receiving and/or transmitting data (e.g., over a network, such as the internet), such as data received by a blood flow monitor and/or from one or more sensors attached to the blood flow monitor (e.g., Doppler sounds, graphic data relating to blood flow). Any suitable remote device can be used to complete a method described herein and selection of a suitable remote device can be based on various considerations, such as the intended use of the remote device and/or the locations desired to position the remote device. Examples of remote devices considered suitable to utilize in a method described herein include mobile devices, such as cellular phones (e.g., smart phones), personal computers, laptops, tablets, devices that include a visual display field, devices that can access patient data obtained by a blood flow monitor (e.g., directly, through a cloud computing resource that the remote device can access) such that a clinician can view the patient data using the remote device (e.g., listen to Doppler sounds, view data), combinations of the devices described herein, and any other device considered suitable for a particular embodiment.

Any suitable Doppler blood flow monitor that has a speaker, can monitor blood flow through a blood vessel, and can be configured to emit Doppler sounds can be used to complete a method of calibrating a blood flow monitoring device. In alternative embodiments, a blood flow monitor can include any software and/or components capable of receiving and/or transmitting data (e.g., over a network, such as the internet). In embodiments in which a blood flow monitor includes software and/or components capable of transmitting data, the blood flow monitor can transmit data associated with blood flow and/or Doppler sounds directly to a remote device or a cloud computing resource that a remote device can access (e.g., via an app). Selection of a suitable blood flow monitor can be based on various considerations, such as the type of blood vessel being monitored and/or the procedure that has been, or is intended to be, completed. Examples of blood flow monitors considered suitable to use in a method described herein include Doppler blood flow monitors, blood flow monitors capable of emitting sounds relating to blood flow through a blood vessel, and any other blood flow monitor considered suitable for a particular embodiment. While the blood flow monitor has been described as emitting Doppler sounds, a monitor included in a method described herein can emit any suitable type of sound that can be utilized to calibrate the sound quality of a remote device.

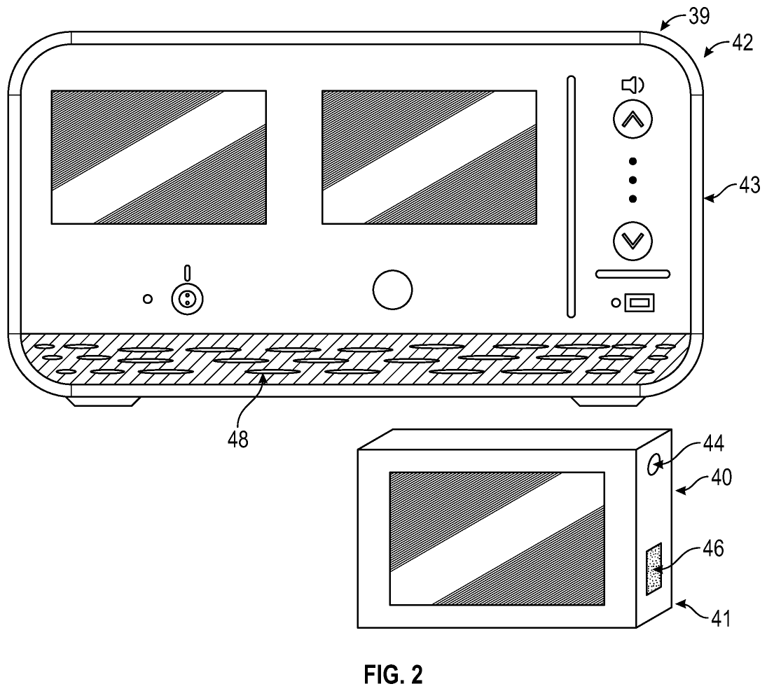

FIG. 2 illustrates an example blood flow monitoring system 39 that includes a remote device 40 and a blood flow monitor 42. The remote device 40 is positioned within physical proximity of the blood flow monitor 42. In the illustrated embodiment, the remote device 40 is a cellular phone 41 and the blood flow monitor 42 is a Doppler blood flow monitor 43. The remote device 40 has a microphone 44 and speaker 46. The Doppler blood flow monitor 42 has a speaker 48 and can obtain patient data relating to blood flow through a blood vessel using one or more sensors, as described in more detail herein.

Step 14 can be accomplished using any suitable technique or method of activating the microphone of the remote device such that the microphone can receive sounds emitted by a speaker of a secondary device (e.g., blood flow monitor 42). For example, in the illustrated embodiment, software and/or hardware in the remote device 40 (e.g., an app installed on the remote device 40) can be activated to place the remote device 40 in a calibration mode and to activate the microphone 44 of the remote device 40.

Step 16 can be accomplished using any suitable technique or method of emitting Doppler sounds through the speaker of the blood flow monitor. For example, in the illustrated embodiment, software and/or hardware in the blood flow monitor 42 can be activated such that Doppler sounds are emitted through the speaker 48 of the blood flow monitor 42. Doppler sounds emitted by a blood flow monitor can be pre-recorded characterized sounds or sounds that relate to patient data (e.g., pre-recorded Doppler sounds from a patient, live Doppler sounds from a patient). In the illustrated embodiment, the Doppler sounds emitted by the blood flow monitor are pre-recorded characterized sounds that allow for calibration of the remote device.

Step 18 can be accomplished using any suitable technique or method of receiving the Doppler sounds as received Doppler sounds using the microphone of the remote device. For example, in the illustrated embodiment, software and/or hardware in the remote device 40 can be activated to save the received Doppler sounds locally and/or on a cloud computing resource that the remote device 40 can access.

Step 20 can be accomplished using any suitable technique or method of emitting the received Doppler sounds through the speaker of the remote device as remote device sounds. For example, in the illustrated embodiment, software and/or hardware in the remote device 40 can be activated such that the received Doppler sounds are emitted through the speaker 46 of the remote device 40 as remote device sounds.

Step 22 can be accomplished using any suitable technique or method of receiving the remote device sounds using a microphone of a remote device. For example, in the illustrated embodiment, software and/or hardware in the remote device 40 can be utilized to save the remote device sounds locally and/or on a cloud computing resource that the remote device 40 can access.

Step 24 can be accomplished by identifying any differences between the received Doppler sounds and the received remote device sounds. Step 24 can be accomplished using any suitable technique or method of identifying any differences between the received Doppler sounds and the received remote device sounds. For example, in the illustrated embodiment, software and/or hardware in the remote device 40 can be utilized to identify any differences between the received Doppler sounds and the received remote device sounds. For example, a frequency response curve can be obtained and digital signal processing (DSP) can be used to filter and gain the signal to compensate for speaker performance. In an alternative embodiment, step 22 can comprise receiving the remote device sounds using a microphone of the blood flow monitor and step 24 can comprise identifying any differences between the Doppler sounds and the received remote device sounds. If differences exist, step 26 can comprise calibrating the blood flow monitor based on the identified differences between the Doppler sounds and the received remote device sounds. If no differences exist, step 28 can comprise completing the method.

Step 26 can be accomplished by calibrating the remote device based on any identified differences between the received Doppler sounds and the received remote device sounds such that when the received Doppler sounds are emitted from the speaker of the remote device (e.g., calibrated remote device) they emulate the Doppler sounds emitted from the blood flow monitor (e.g., the received Doppler sounds are the same as, or substantially the same as, the Doppler sounds). This can be accomplished using software and/or hardware in the remote device 40. For example, step 26 can be accomplished by adjusting the microphone, digital signal processing (DSP), treble, mid, base, and/or tone settings of the remote device and/or using sound equalization characterization techniques to adjust the data received from the microphone and/or the data sent to the speaker of the remote device. Alternatively, calibration can be completed by a clinician via self-calibration, or self-calibration can be accomplished in combination with the software and/or hardware calibration described herein. Optionally, step 14, step 16, step 18, step 20, step 22, step 24, and/or step 26 can be repeated to confirm proper calibration of the remote device and/or to further calibrate the remote device. Any of the steps described herein as being completed by a remote device and/or blood flow monitor can optionally be completed using software and/or hardware in the remote device 40 (e.g., an app installed on the remote device 40) and/or on the blood flow monitor.

In an alternative embodiment, step 12 can be omitted and step 14 can comprise placing the remote device in a calibration mode such that the microphone of the remote device is active and can receive Doppler sounds emitted by the speaker of the remote device. In this alternative embodiment, step 16 comprises emitting Doppler sounds through the speaker of the remote device and the remainder of the method can be completed as described. This alternative method allows a user to calibrate a remote device without having to be in physical proximity to a blood flow monitor.

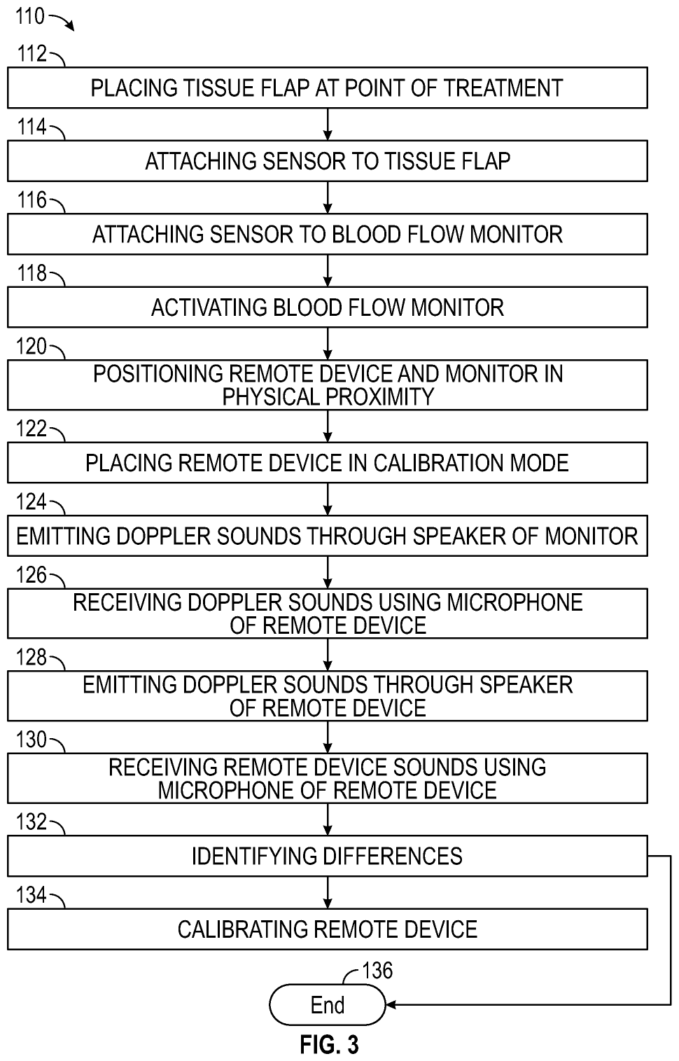

FIG. 3 is a schematic illustration of another example method 110 of calibrating a blood flow monitoring device.

A step 112 comprises placing a tissue flap at a point of treatment. The tissue flap includes a section of tissue that includes a blood vessel. Another step 114 comprises attaching a sensor to the tissue flap to monitor blood flow through the blood vessel. The sensor is attached to the tissue flap such that it contacts the blood vessel. Another step 116 comprises attaching the sensor to a blood flow monitor that has a speaker, a visual display field, and is configured to emit Doppler sounds. Another step 118 comprises activating the blood flow monitor such that the blood flow monitor is monitoring blood flow through the blood vessel. Another step 120 comprises positioning a remote device and the blood flow monitor within physical proximity of one another. The remote device has microphone and a speaker. Another step 122 comprises placing the remote device in a calibration mode such that the microphone of the remote device is active and can receive Doppler sounds relating to the blood flow through the blood vessel emitted by the speaker of the blood flow monitor. Another step 124 comprises emitting Doppler sounds relating to the blood flow through the blood vessel through the speaker of the blood flow monitor. Another step 126 comprises receiving the Doppler sounds relating to the blood flow through the vessel as received Doppler sounds using the microphone of the remote device. Another step 128 comprises emitting the received Doppler sounds through the speaker of the remote device as remote device sounds. Another step 130 comprises receiving the remote device sounds using the microphone of the remote device. Another step 132 comprises identifying any differences between the received Doppler sounds and the received remote device sounds. If differences exist, another step 134 comprises calibrating the remote device based on the identified differences between the received Doppler sounds and the received remote device sounds. If no differences exist, another step 136 comprises completing the method.

Step 112 can be accomplished by placing the tissue flap at any suitable point of treatment and selection of a suitable point of treatment can be based on various considerations, such as the treatment intended to be performed. Examples of points of treatment considered suitable to place a tissue flap include the head (e.g., face), neck, chest, breast, back, abdomen, arms, buttocks, legs (e.g., thighs), areas of a body that previously included defects, such as those from an injury or other surgery (e.g., mastectomy), and any other point of treatment considered suitable for a particular embodiment.

A tissue flap utilized in a method described herein can comprise any suitable section of tissue that can be used to complete an autologous tissue reconstruction procedure, includes at least one blood vessel, and/or that has been moved from a donor site of a patient to a recipient site of the patient. Examples of tissue flaps considered suitable to complete a method described herein include local flaps (e.g., advancement flaps, rotation flaps, transposition flaps, interpolation flaps), free flaps, transverse rectus abdominis muscle flaps, deep inferior epigastric perforator flaps, latissimus dorsi flaps, gluteal artery perforator flaps, transverse upper gracilis flaps, flaps obtained from the chest, breast, back, abdomen, arms, buttocks, or legs (e.g., thighs) of a patient, combinations of those described herein, and any other tissue flap considered suitable for a particular embodiment.

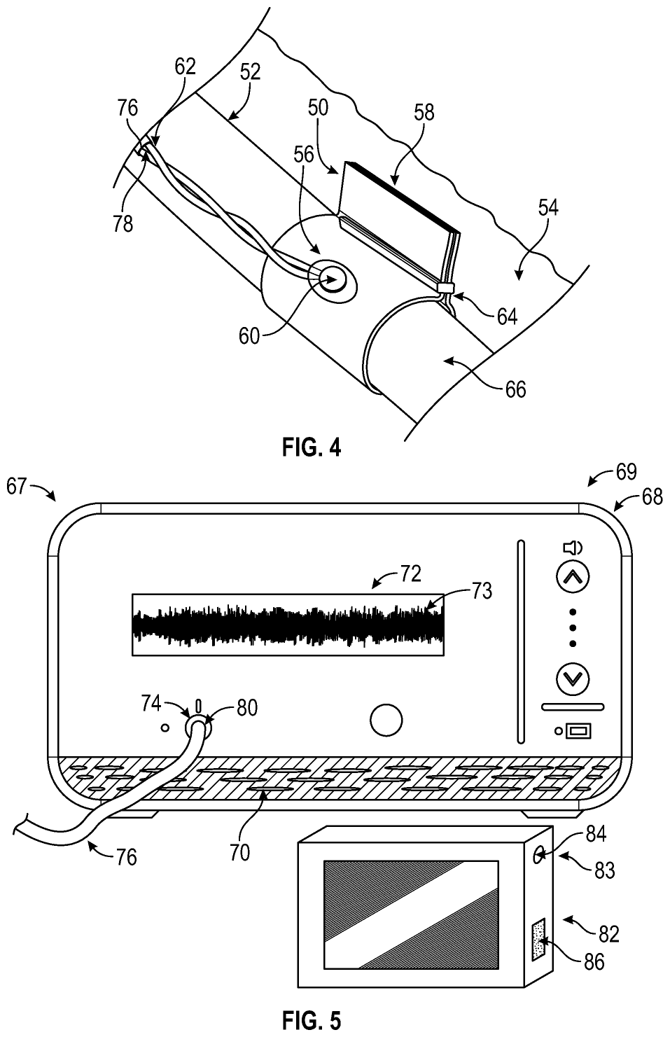

Step 114 can be accomplished by attaching any suitable sensor to the tissue flap such that the sensor contacts the blood vessel. FIG. 4 illustrates a sensor 50 contacting a blood vessel 52 of a tissue flap 54 (e.g., directly contacting the blood vessel 52). In the illustrated embodiment, the sensor 50 is a Doppler probe 56 that includes a cuff 58, a probe crystal 60 attached to the cuff 58, and a wire member 62 attached to the probe crystal 60. The sensor 50 is attached to the blood vessel 52 by wrapping the cuff 58 around the blood vessel 52 and attaching the cuff 58 directly to the blood vessel 52 (e.g., surgically) using a surgical clip 64. The sensor 50 allows for monitoring of blood flow within the blood vessel 52 intraoperatively and postoperatively following completion of a procedure (e.g., reconstructive procedure). In the illustrated embodiment, the sensor 50 is disposed deep within the tissue flap 54 (i.e., beneath a surface of the flap 54, subcutaneously) and is attached to a vein 66. However, in alternative embodiments, a sensor can be positioned at any suitable location on, or within, a tissue flap and can be attached to any suitable blood vessel, such as an artery.

While the sensor 50 has been illustrated as a Doppler probe 56, a sensor used to complete a method described herein can include any suitable sensor capable of monitoring blood flow through a blood vessel and selection of a suitable sensor can be based on various considerations, such as the type of tissue flap being used to complete the method. Examples of sensors considered suitable to use as a sensor include in vivo sensors, ultrasonic probes, electromagnet probes, Doppler probes, such as Cook-Swartz Doppler probes, sensors that include components capable of transmitting and/or receiving data wirelessly, combinations of the sensors described herein, and any other sensor considered suitable for a particular embodiment. While the sensor 50 has been illustrated as including various components and as being attached to the blood vessel 52 in a particular manner, a sensor can include any suitable number of components and be attached to a blood vessel in any suitable manner. Selection of a suitable number of components for a sensor to include and of a suitable method or technique to attach a sensor to a blood vessel can be based on various considerations, including the material forming the sensor and/or the size and/or location of the blood vessel.

Step 116 can be accomplished by attaching the sensor to any suitable blood flow monitor that has a speaker, a visual display field, and is configured to emit Doppler sounds. FIG. 5 illustrates an example blood flow monitoring system 67 that includes a blood flow monitor 68 and a remote device 82. The blood flow monitor 68 has a speaker 70, a visual display field 72, and a first channel port 74. The blood flow monitor 68 is moveable between an off state and an on state. FIG. 5 illustrates the blood flow monitor 68 in the on state. In the illustrated embodiment, the sensor 50 is attached to the blood flow monitor 68 using an extension cable 76 that has a first end 78 attached to the sensor 50 and a second end 80 attached to the blood flow monitor 68 (e.g., first channel port 74). However, in alternative embodiments, a sensor can be attached directly to a blood flow monitor without using an extension cable.

While the blood flow monitor 68 has been illustrated as having a particular number of visual display fields (e.g., discrete portions of a display, discrete displays) and as having a particular number of channel ports, a blood flow monitor can include any suitable number of visual display fields, channel ports, and other components, features, and/or devices. Selection of a suitable number of visual display fields and/or channel ports to include in a blood flow monitor can be based on various considerations, including the number of sensors being utilized to monitor a tissue flap. Examples of numbers of visual display fields and/or channel ports considered suitable to include in a blood flow monitor include one, at least one, two, a plurality, three, four, five, more than five, and any other number considered suitable for a particular embodiment. Examples of other components, features, and/or devices considered suitable to include in a blood flow monitor include those described herein, one or more amplifiers, and any other component considered suitable for a particular embodiment. Depending on the type of sensors being utilized, a blood flow monitor can also include any software and/or components capable of receiving and/or transmitting data (e.g., over a network, such as the internet). In embodiments in which a blood flow monitor includes software and/or component capable of receiving and/or transmitting data, the blood flow monitor can receive data wirelessly from one or more sensors and/or can transmit data associated with blood flow and/or Doppler sounds directly to a remote device or a cloud computing resource that a remote device can access (e.g., via an app) such that data received by one or more sensors and/or data displayed within a visual display field can be provided to the remote device and/or a clinician.

Step 118 can be accomplished by moving the blood flow monitor from an off state to an on state to observe the blood flow through the blood vessel. FIG. 5 illustrates the blood flow monitor 68 in the on state. The visual display field 72 shows data (e.g., sound data from Doppler probe 56 in graphical form) received from the sensor 50 relating to blood flow over time 73 (e.g., time domain, amplitude over time, average amplitude over time) for the blood vessel 52. In the illustrated embodiment, the visual display field 72 shows a graphic, or a visual representation of data in graphical nature. The visual display field 72 shows a graphic of the blood flow over time 73 (e.g., sound graphic, visual representation of blood flow velocity in graphical nature). The data provided in a visual display field can be shown in real-time, be held or frozen, or a clinician can select a window of time (e.g., adjustable, clinician defined) within which the clinician would like to review data. Inclusion of the visual display field 72 is considered advantageous at least because it allows a clinician to review, or monitor, trend data regarding blood flow over time and tissue health information. For example, the visual display field 72 allows a clinician to review, or monitor, trend data regarding blood flow over time.

While the visual display field 72 has been illustrated as displaying particular data, a visual display field can illustrate any suitable data and selection of suitable data to display on a visual display field can be based on various considerations, including the data desired by a clinician relating to an implanted tissue flap. Examples of data considered suitable to display on a visual display field included on a blood flow monitor include data relating to blood flow of a blood vessel included in a tissue flap, tissue health, tissue oxygenation saturation levels, temperature, blood pressure, orifice diameter (e.g., artificial, original), any data over a period of time, combinations of the data described herein, and any other data considered suitable for a particular embodiment.

While the visual display field 72 has been illustrated as showing a single data set (e.g., blood flow over time) within a single viewing area within the display field, a visual display field can alternatively show multiple data sets, each within a separate viewing area. Optionally, a visual display field can include trend lines, such as average amplitude over time, relating to the data displayed within a visual display field. Furthermore, a blood flow monitor can optionally allow a clinician to record, freeze, and/or store data displayed on a visual display field and/or data obtained by a sensor (e.g., over a period of time (e.g., 15 minutes)) within a local storage device included in the blood flow monitor and/or remotely.

A speaker included in a blood flow monitor can emit any suitable sound, such as Doppler sounds provided by a sensor to allow a clinician to listen to sounds relating to historical and/or current blood flow through a blood vessel of a tissue flap. In addition, a blood flow monitor can provide various alerts to provide feedback to a clinician, such as sound alerts associated with clinician-defined settings, clinician-defined trigger points (e.g., change in sound, change in a device event, change in sensor readings), alerts relating to a pre-defined sensor reading, alerts relating to a pre-defined tissue oxygenation saturation level, alerts relating to a pre-defined blood pressure level, alerts relating to a pre-defined orifice diameter, alters relating to the status of the blood flow monitor (e.g., in off state, lost power, malfunctioning), and any other alert considered suitable for a particular embodiment.

Step 120 can be accomplished as described herein with respect to step 12. FIG. 5 illustrates an example remote device 82 positioned within physical proximity of blood flow monitor 68. In the illustrated embodiment, the remote device 82 is a cellular phone 83 and the blood flow monitor 68 is a Doppler blood flow monitor 69. The remote device 82 has a microphone 84 and speaker 86. The Doppler blood flow monitor 69 has a speaker 70 and can obtain patient data relating to blood flow through a blood vessel using one or more sensors, as described in more detail herein.

Step 122 can be accomplished as described herein with respect to step 14. Step 124 can be accomplished as described herein with respect to step 16. Step 126 can be accomplished as described herein with respect to step 18. Step 128 can be accomplished as described herein with respect to step 20. Step 130 can be accomplished as described herein with respect to step 22. Step 132 can be accomplished as described herein with respect to step 24. Step 134 can be accomplished as described herein with respect to step 26. Step 136 can be accomplished as described herein with respect to step 28.

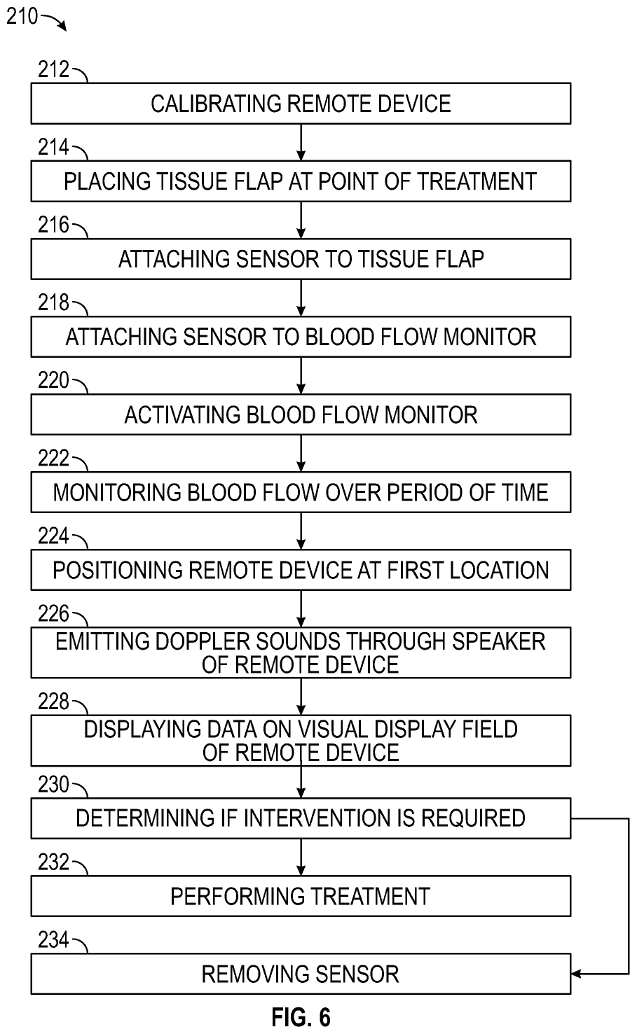

FIG. 6 is a schematic illustration of an example method 210 of monitoring blood flow using a calibrated blood flow monitoring device.

A step 212 comprises calibrating the sound quality of a remote device that has a speaker, a microphone, and a visual display field. Another step 214 comprises placing a tissue flap at a point of treatment. The tissue flap includes a section of tissue that includes a blood vessel. Another step 216 comprises attaching a sensor to the tissue flap to monitor blood flow through the blood vessel. The sensor is attached to the tissue flap such that it contacts the blood vessel. Another step 218 comprises attaching the sensor to a blood flow monitor that has a speaker, a visual display field, and is configured to emit Doppler sounds. Another step 220 comprises activating the blood flow monitor such that the blood flow monitor is monitoring blood flow through the blood vessel. Another step 222 comprises monitoring the blood flow through the blood vessel over a period of time. Another step 224 comprises positioning the remote device at a first location that is different from a second location at which the blood flow monitor is disposed. Another step 226 comprises emitting Doppler sounds relating to the blood flow through the blood vessel through the speaker of the remote device. Another step 228 comprises displaying data (e.g., sound data from Doppler probe in graphical form) received from the sensor relating to blood flow over time (e.g., time domain, amplitude over time, average amplitude over time) for the blood vessel on the visual display field of the remote device. Another step 230 comprises determining if the blood flow through the blood vessel (e.g., Doppler sounds, data provided on visual display field) indicates intervention is required. If intervention is required, another step 232 comprises performing treatment to accomplish the intervention. If intervention is not required, another step 234 comprises removing the sensor from the tissue flap.

Step 212 can be accomplished by completing steps 12 through 28, as described herein. Step 212 is completed prior to step 214 when the Doppler sounds being emitted from the blood flow monitor are pre-recorded characterized sounds. Step 212 can alternatively be completed subsequent to step 220 in embodiments in which the Doppler sounds relate to patient data (e.g., pre-recorded Doppler sounds from a patient, live Doppler sounds from a patient).

Step 214 can be accomplished as described herein with respect to step 112. Step 216 can be accomplished as described herein with respect to step 114. Step 218 can be accomplished as described herein with respect to step 116. Step 220 can be accomplished as described herein with respect to step 118.

Step 222 can be accomplished by maintaining the blood flow monitor in the on state such that data received from the sensor relating to the blood flow through the blood vessel can be recorded, translated, and/or provided via the visual display field and/or transmitted to a remote device or a cloud computing resource that a remote device can access (e.g., via an app) such that data associated with blood flow and/or Doppler sounds can be monitored by a clinician.

A blood flow monitor can be maintained in the on state for any suitable period of time to monitor blood flow through a blood vessel of a tissue flap and selection of a suitable period of time can be based on various considerations, such as the location of the implant site of a tissue flap and/or the type of tissue flap that has been implanted. Examples of periods of time considered suitable to maintain a blood flow monitor in an on state to monitor blood flow through a blood vessel of a tissue flap include one or more seconds, one or more minutes, one or more hours, one or more days, one or more weeks, and any other period of time considered suitable for a particular embodiment. For example, a blood flow monitor can be maintained in an on state to monitor blood flow through a blood vessel of a tissue flap for about 24 hours, about 48 hours, about 72 hours, and any other period of time considered suitable for a particular embodiment.

Step 224 can be accomplished using any suitable mode of transportation, such as walking, driving, and/or flying. A first location can be any location at which the clinician using a remote device is disposed, such as an office or a residence. A second location can be any location at which the blood flow monitor is disposed, such as within a patient room, a hospital, or an operating room. Optionally, step 224 can be omitted from method 210.

Step 226 can be accomplished using any suitable technique or method of emitting the Doppler sounds through the speaker of the remote device. For example, in the illustrated embodiment, software and/or hardware in the remote device can be activated such that the Doppler sounds (e.g., data received from the sensor is emitted from the speaker of the remote device) are emitted through the speaker of the remote device.

Step 228 can be accomplished using any suitable technique or method of displaying data received from the sensor relating to blood flow over time for the blood vessel on a visual display field of the remote device. For example, in the illustrated embodiment, software and/or hardware in the remote device can be activated such that the data received from the sensor is displayed on a visual display field of the remote device. Optionally step 228 can be omitted from method 210.

Step 230 can be accomplished by reviewing the data provided through the speaker of the remote device and/or provided on the visual display field of the remote device to determine whether any of the data indicates that the tissue flap requires intervention. For example, if the data displayed in a visual display field (e.g., blood flow over time) and/or the Doppler sounds emitted through the speaker of the remote device indicate that the data is below a threshold (e.g., clinician defined), intervention is required. Alternatively, if the data displayed in a visual display field (e.g., blood flow over time) and/or the Doppler sounds emitted through the speaker of the remote device indicate that the data is above the threshold, intervention is not required.

If intervention is required, step 232 can be accomplished by performing any suitable treatment to accomplish the intervention. Examples of treatments considered suitable to accomplish intervention include repositioning a sensor, replacing an implanted tissue flap with a second tissue flap, repositioning the patient, correcting a kink, twist, or tension on a vessel, correcting the design of the anastomosis, treating infection, clot extraction, administering one or more drugs (e.g., anticoagulants, blood thinners), increasing blood pressure, combinations of the treatments described herein, and any other treatment considered suitable for a particular embodiment. If intervention is not required, step 234 can be accomplished by removing the sensor from the blood vessel of the tissue flap. Removal of the sensor can be accomplished by removing any sutures and/or tape from the wire member outside of the body of the patient and applying an axial force on the wire member near the crystal and away from the patient to disengage the crystal from the cuff. The cuff can optionally be left in place around the blood vessel and any opening can be closed (e.g., using sutures). Alternatively, the cuff can be removed from the blood vessel.

In alternative embodiments, and depending on the type of sensor being used to provide data to a blood flow monitor, step 232 and/or step 234 can be omitted from method 210. Step 234 can be omitted, for example, in instances in which the sensor is biodegradable and/or provides data to a blood flow monitor wirelessly. An optional step comprises deactivating the blood flow monitor, which can be accomplished by moving the blood flow monitor from the on state to the off state.

The methods described herein are considered advantageous at least because they allow clinicians to remotely evaluate patient data (e.g., tissue flap, blood flow) in real time using live data, or using recordings, received from a blood flow monitor (e.g., via software, such as an app installed on a mobile computing device) using a calibrated remote device that can emulate Doppler sounds emitted from the blood flow monitor. Generally, the sound quality of a blood flow monitoring device is dependent on the specific device from which the sounds relating to blood flow (e.g., Doppler sounds) are being played. Therefore, clinicians are trained and accustomed to hearing a certain sound quality from particular blood flow monitors. Using a remote device that has been calibrated, as described herein, such that it emulates Doppler sounds as close to the same sound quality as Doppler sounds emitted by the blood flow monitor from which the Doppler sounds have been obtained, increases the reliability of the sound quality of the Doppler sounds being emitted by the remote device and provides a mechanism for clinicians to more accurately analyze the health and status of a blood vessel and/or tissue flap remotely. As described herein, a remote device can be calibrated by, among other steps, positioning a remote device and a blood flow monitor in physical proximity of one another, identifying the differences between Doppler sounds and remote device sounds received by the remote device, and calibrating the remote device based on the identified differences between the received Doppler sounds and the received remote device sounds. This process can be completed any number of times and relative to any type of blood flow monitor. As a result, clinicians can easily calibrate a remote device to remotely monitor the health and status of a blood vessel and/or tissue flap, which frees up time to perform other tasks.

Those with ordinary skill in the art will appreciate that various modifications and alternatives for the described and illustrated embodiments can be developed in light of the overall teachings of the disclosure, and that the various elements and features of one example described and illustrated herein can be combined with various elements and features of another example without departing from the scope of the invention. Accordingly, the particular arrangement of elements and steps disclosed herein have been selected by the inventor(s) simply to describe and illustrate examples of the invention and are not intended to limit the scope of the invention or its protection, which is to be given the full breadth of the appended claims and any and all equivalents thereof.

What is claimed is:

1. A method of calibrating a blood flow monitoring device comprising:
positioning a remote device and a blood flow monitor within physical proximity of one another, the remote device having a microphone and a speaker, the blood flow monitor having a speaker and configured to emit Doppler sounds;
placing the remote device in a calibration mode such that the microphone of the remote device is active and can receive Doppler sounds emitted by the speaker of the blood flow monitor;
emitting Doppler sounds through the speaker of the blood flow monitor;
receiving the Doppler sounds as received Doppler sounds using the microphone of the remote device;
emitting the received Doppler sounds through the speaker of the remote device as remote device sounds;
receiving the remote device sounds using the microphone of the remote device;
identifying any differences between the received Doppler sounds and the received remote device sounds; and
calibrating the remote device based on the identified differences between the received Doppler sounds and the received remote device sounds.

2. The method of claim 1, wherein calibrating the remote device based on the identified differences between the received Doppler sounds and the received remote device sounds is accomplished using sound equalization characterization techniques.

3. The method of claim 1, wherein the Doppler sounds are pre-recorded characterized sounds.

4. The method of claim 1, wherein the Doppler sounds relate to patient data.

5. The method of claim 4, wherein the Doppler sounds relate to blood flow through a vein.

6. The method of claim 4, wherein the Doppler sounds relate to blood flow through an artery.

7. The method of claim 1, wherein receiving the Doppler sounds as received Doppler sounds using the microphone of the remote device is accomplished such that the received Doppler sounds are saved locally to the remote device.

8. The method of claim 1, wherein receiving the remote device sounds using the microphone of the remote device is accomplished such that the received remote device sounds are saved locally to the remote device.

9. A method of calibrating a blood flow monitoring device comprising:
placing a tissue flap at a point of treatment, the tissue flap including a section of tissue that includes a blood vessel;
attaching a sensor to the tissue flap to monitor blood flow through the blood vessel, the sensor attached to the tissue flap such that it contacts the blood vessel;
attaching the sensor to a blood flow monitor having a speaker and configured to emit Doppler sounds;
activating the blood flow monitor such that the blood flow monitor is monitoring blood flow through the blood vessel;
positioning a remote device and the blood flow monitor within physical proximity of one another, the remote device having a microphone and a speaker;
placing the remote device in a calibration mode such that the microphone of the remote device is active and can receive Doppler sounds relating to the blood flow through the blood vessel emitted by the speaker of the blood flow monitor;
emitting Doppler sounds relating to the blood flow through the blood vessel through the speaker of the blood flow monitor;
receiving the Doppler sounds relating to the blood flow through the blood vessel as received Doppler sounds using the microphone of the remote device;
emitting the received Doppler sounds through the speaker of the remote device as remote device sounds;
receiving the remote device sounds using the microphone of the remote device;
identifying any differences between the received Doppler sounds and the received remote device sounds; and
calibrating the remote device based on the identified differences between the received Doppler sounds and the received remote device sounds.

10. The method of claim 9, wherein calibrating the remote device based on the identified differences between the received Doppler sounds and the received remote device sounds is accomplished using sound equalization characterization techniques.

11. The method of claim 9, wherein the blood vessel is a vein.

12. The method of claim 9, wherein the blood vessel is an artery.

13. The method of claim 9, wherein receiving the Doppler sounds relating to the blood flow through the blood vessel as received Doppler sounds using the microphone of the remote device is accomplished such that the received Doppler sounds are saved locally to the remote device.

14. The method of claim 9, wherein receiving the remote device sounds using the microphone of the remote device is accomplished such that the received remote device sounds are saved locally to the remote device.

15. The method of claim 9, wherein the remote device has a visual display field; and
wherein the blood flow monitor has a visual display field.

16. The method of claim 15, further comprising displaying data relating to the blood flow through the blood vessel on the visual display field of the blood flow monitor; and
further comprising displaying data relating to the blood flow through the blood vessel on the visual display field of the remote device.

17. A method of monitoring blood flow comprising:
calibrating the sound quality of a remote device having a speaker and a microphone;
placing a tissue flap at a point of treatment, the tissue flap including a section of tissue that includes a blood vessel;
attaching a sensor to the tissue flap to monitor blood flow through the blood vessel, the sensor attached to the tissue flap such that it contacts the blood vessel;
attaching the sensor to a blood flow monitor having a speaker and configured to emit Doppler sounds;
activating the blood flow monitor such that the blood flow monitor is monitoring blood flow through the blood vessel;
monitoring the blood flow through the blood vessel over a period of time;
positioning the remote device at a first location that is different from a second location at which the blood flow monitor is disposed;
emitting Doppler sounds relating to the blood flow through the blood vessel through the speaker of the remote device; and
determining if the Doppler sounds relating to the blood flow through the blood vessel indicate intervention is required, if intervention is required the method further comprises performing treatment to accomplish the intervention, if intervention is not required the method further comprises removing the sensor from the tissue flap.

18. The method of claim 17, wherein the remote device has a visual display field.

19. The method of claim 18, further comprising displaying data relating to the blood flow through the blood vessel on the visual display field of the remote device.

20. The method of claim 19, wherein the data relating to the blood flow through the blood vessel displayed on the visual display field of the remote device is sound data from the sensor in graphical form.
